(19) United States
(12) Patent Application Publication
(10) Pub. No.: US 2025/0012187 A1
(43) Pub. Date: Jan. 9, 2025

(54) ZERO-FLARING WELL TESTING SYSTEMS AND METHODS

(71) Applicant: Schlumberger Technology Corporation, Sugar Land, TX (US)

(72) Inventor: Mikhail Kydatov, Atyrau (KZ)

(21) Appl. No.: 18/697,939

(22) PCT Filed: Oct. 26, 2021
(86) PCT No.: PCT/US2021/072044

§ 371 Date: Apr. 2, 2024

(51) Int. Cl. E21B 49/08; E21B 33/03; E21B 43/40; G01N 33/24

(52) U.S. Cl. CPC E21B 49/086; E21B 33/03; E21B 43/40; G01N 33/24

(57) ABSTRACT

A zero-flaring well testing assembly is provided. The zero-flaring well testing assembly can be coupled to receive a gas-containing well effluent from a wellhead assembly. The zero-flaring well testing assembly can include a flowmeter and at least one multiphase pump. The zero-flaring well testing assembly can also be coupled to a production line to provide at least a gas-containing portion of the well effluent to the production line without flaring gas of the well effluent. Additional systems, methods, and devices are also disclosed.

### BACKGROUND

This section is intended to introduce the reader to various aspects of art that may be related to various aspects of the presently described embodiments. This discussion is believed to be helpful in providing the reader with background information to facilitate a better understanding of the various aspects of the present embodiments. Accordingly, it should be understood that these statements are to be read in this light, and not as admissions of prior art.

Wells are generally drilled into subsurface rocks to access fluids, such as hydrocarbons, stored in subterranean formations. The subterranean fluids can be produced from these wells through known techniques. Operators may want to know certain characteristics of produced fluids to facilitate efficient and economic exploration and production. For example, operators may want to know flow rates of produced fluids. These produced fluids are often multiphase fluids (e.g., those having some combination of water, oil, and gas), making measurement of the flow rates more complex. Surface well testing provides various information about the reservoir and its fluids, such as volumetric flow rates of fluids produced from a well and properties of the produced fluids.

Surface well testing equipment may be temporarily installed at a wellsite for well test operations. The surface well testing equipment can include a separator that facilitates separation of the multiphase fluid, such as into gas, water, and oil phases. In some instances, separated hydrocarbons may be burned at the wellsite. For instance, an oil burner may be used to burn separated oil and a gas flare may be used to burn separated gas.

### SUMMARY

Certain aspects of some embodiments disclosed herein are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

Certain embodiments of the present disclosure generally relate to wellsite operations. More specifically, some embodiments relate to a zero-flaring well testing assembly for receiving fluid from a well, measuring a characteristic of the fluid, and injecting the fluid from the zero-flaring well testing assembly into an output line without flaring gas from the fluid. In some embodiments, the zero-flaring well testing assembly includes one or more multiphase pumps for boosting pressure of a received well effluent. When multiple multiphase pumps are used, these pumps may be connected in series or in parallel. A liquid, such as water, can be added to the well effluent upstream of the pumps to lower the gas volume fraction (GVF) of the well effluent received in the pumps. In certain embodiments, the liquid added to the well effluent upstream of the pumps is water separated from the well effluent with a separator of the zero-flaring well testing assembly. A flowmeter, such as a multiphase flowmeter, may be used to monitor the GVF and other parameters of the well effluent.

Various refinements of the features noted above may exist in relation to various aspects of the present embodiments. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the brief summary presented above is intended only to familiarize the reader with certain aspects and contexts of some embodiments without limitation to the claimed subject matter.

### BRIEF DESCRIPTION OF THE DRAWINGS

These and other features, aspects, and advantages of certain embodiments will become better understood when the following detailed description is read with reference to the accompanying drawings in which like characters represent like parts throughout the drawings, wherein:

FIG. 1 generally depicts a well testing assembly deployed at a wellsite in accordance with an embodiment of the present disclosure;

FIG. 2 is a schematic of an apparatus with a zero-flaring well testing assembly having pumps, a flowmeter, a separator, and a recirculation line in accordance with one embodiment;

FIG. 3 is a flowchart representing a process for using a zero-flaring well testing assembly at the wellsite in accordance with one embodiment;

FIG. 4 is a schematic of an apparatus with a zero-flaring well testing assembly having two pumps connected in series, a flowmeter, a separator, and a recirculation line in accordance with one embodiment;

FIG. 5 is a schematic of an apparatus with a zero-flaring well testing assembly having two pumps connected in series, a flowmeter, a separator, a slug catcher, and a recirculation line in accordance with one embodiment;

FIG. 6 is a schematic of an apparatus with a zero-flaring well testing assembly having two pumps connected in parallel, a flowmeter, a separator, a slug catcher, and a recirculation line in accordance with one embodiment;

FIG. 7 is a schematic of an apparatus with a zero-flaring well testing assembly in another configuration having a flowmeter and pumps with other components in accordance with one embodiment;

FIG. 8 generally depicts components of the zero-flaring well testing assembly integrated into a mobile package on a trailer to facilitate movement of the assembly to a wellsite in accordance with one embodiment; and

FIG. 9 is a block diagram of components of a programmable controller for operation of a zero-flaring well testing assembly in accordance with one embodiment.

### DETAILED DESCRIPTION OF SPECIFIC EMBODIMENTS

Specific embodiments of the present disclosure are described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time-consuming, but would nevertheless be a routine undertaking of design, fabrication, and manufacture for those of ordinary skill having the benefit of this disclosure.

When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements. Moreover, any use of “top,” “bottom,” “above,” “below,” other directional terms, and variations of these terms is made for convenience, but does not require any particular orientation of the components.

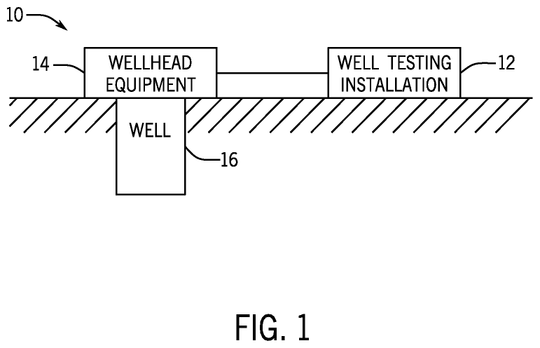

Turning now to the drawings, a wellsite 10 is generally depicted in FIG. 1 in accordance with one embodiment. As presently shown, a well testing installation or assembly 12 is deployed at the wellsite 10 and is coupled to wellhead equipment 14 installed at a well 16. The wellhead equipment 14 can include any suitable components, such as casing and tubing heads and a Christmas tree (e.g., a production tree or test tree), to name but a few examples. Well effluent produced from the well 16 can be routed through the wellhead equipment 14 and into the well testing assembly 12. It will be appreciated that the wellsite 10 can be onshore or offshore. In offshore contexts, the well testing assembly 12 can be installed on an offshore drilling rig at the wellsite 10. In addition to testing, the well testing assembly 12 can be used for well clean-up before putting the well 16 into production. When no longer needed, the well testing assembly 12 may be disconnected from the well 16 and removed from the wellsite 10.

As generally noted above, gas separated from the well effluent in a well testing assembly can be flared off. Burning this gas may have undesirable impacts, however, such as wasting energy and releasing carbon dioxide. At least some embodiments of the present technique include a zero-flaring well testing assembly in which the well effluent is processed without flaring gas from the well effluent. Instead of burning, the gas can be pumped to a production facility or into a well, for instance. And avoiding flaring of the gas may provide various environmental and commercial benefits.

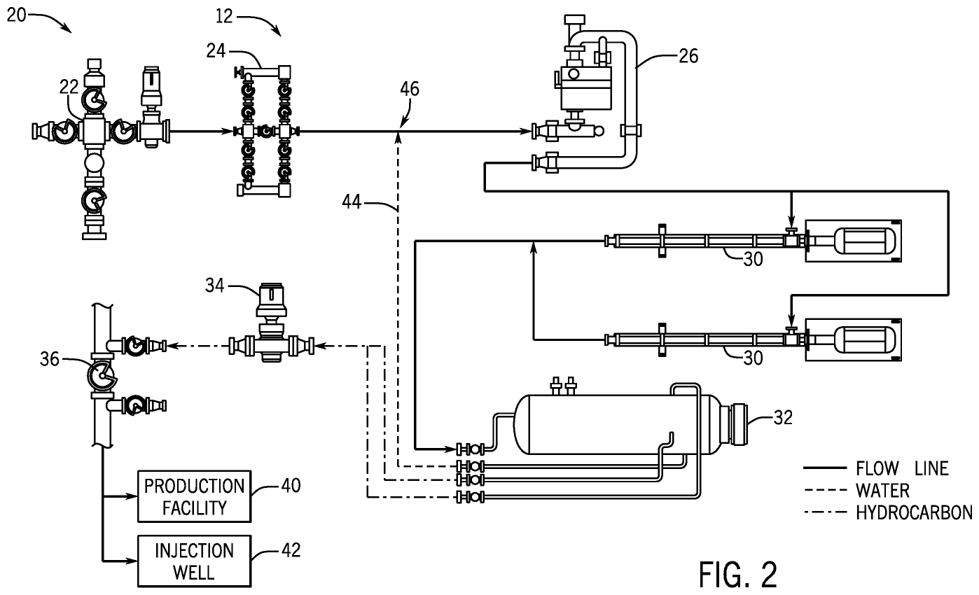

One example of an apparatus 20 with a zero-flaring well testing assembly 12 at a wellsite is generally depicted in FIG. 2. In this depicted embodiment, the apparatus 20 includes a wellhead assembly having a tree 22 mounted over a well (e.g., well 16 of FIG. 1). A manifold 24 (e.g., a choke manifold) controls flow of well effluent from the tree 22 into a well testing assembly that is presently shown to include a flowmeter 26, pumps 30, and a separator 32. The zero-flaring well testing assembly 12 can receive the well effluent from the well via the tree 22, increase pressure of the well effluent via pumps 30, and convey at least a portion of the well effluent to an output line 36 through a valve 34. In some instances, the output line 36 is a production line that carries fluid received from the zero-flaring well testing assembly 12 (e.g., via a production manifold) to a production facility 40 or some other remote facility. In some other instances, fluid in the output line 36 can be injected into a well, such as an injection well 42 or the well 16 from which the effluent was produced.

As will be appreciated, the well effluent can include gas, oil, and water. Gas in the well effluent received by the zero-flaring well testing assembly 12 can be routed into the output line 36 rather than being flared. As such, a gas flaring device can be omitted from the apparatus 20 and the wellsite. Further, in at least some embodiments the zero-flaring well testing assembly 12 provides both gas hydrocarbons and liquid hydrocarbons to the output line 36 rather than burning these hydrocarbons. Thus, an oil burner may also be omitted from the apparatus 20 and the wellsite. Although the zero-flaring well testing assembly 12 can be configured so that gas in the well effluent is routed into the output line 36 without flaring any gas during normal operation, in some instances a gas flaring device can be included in the zero-flaring well testing assembly 12 or elsewhere at the wellsite to enable gas flaring in exceptional circumstances (e.g., for safety reasons). For example, during system overpressure conditions gas is vented through pressure safety valves (to reduce system pressure) and then flared in certain embodiments. Thus, in some instances hydrocarbons of the well effluent are routed through the zero-flaring well testing assembly 12 to the output line 36 without burning any of the hydrocarbons. In some other instances, however, less than all (e.g., a majority) of the hydrocarbons of the well effluent are routed through a well testing assembly 12 to the output line 36 (e.g., during normal operation), while some remaining amount of the hydrocarbons of the well effluent can be burned at the wellsite.

The zero-flaring well testing assembly 12 is shown in FIG. 2 as having two pumps 30 connected in parallel for pumping well effluent through the assembly 12 and into the output line 36. But in other instances, the zero-flaring well testing assembly 12 can include a different number of pumps 30, which may be a single pump 30, three or four pumps 30, or an even greater number of pumps 30. When having multiple pumps 30, these pumps 30 may be connected in parallel (such as shown in FIG. 2), in series, or both (with some pumps connected in series and others in parallel).

The pumps 30 impart energy to the fluid stream within the zero-flaring well testing assembly 12, which can enable delivery of the fluid stream to a distant processing facility (e.g., production facility 40) from remote locations. The pumps 30 can also draw down on the wellhead pressure of well 16, acting as a surface artificial lift method to increase production.

In at least some embodiments, the pumps 30 are multiphase pumps for pumping a multiphase well effluent having both a gaseous component, such as natural gas, and a liquid component, such as one or both of water or liquid hydrocarbon (e.g., crude oil or condensate). Although other types of multiphase pumps could be used, such as positive displacement pumps, in FIG. 2 the depicted pumps 30 are each a motor-driven multistage centrifugal helicoaxial downhole-type pump, which may be mounted horizontally on a rigid skid. One example of such a pump 30 that may be used in the zero-flaring well testing assembly 12 is the REDA HPS™ horizontal multistage surface pumping system produced by Schlumberger Ltd.

In some embodiments, each multiphase pump 30 includes 30-80 stages (e.g., 30 stages, 40 stages, or 75 stages) and has an operating frequency of 35-65 Hz. The volumetric flow rate of the pump 30 may vary depending on size and configuration. By way of example, the pump 30 is a multiphase pump with a volumetric flow rate (at 60 Hz at inline condition for a multiphase mixture) of 5,000-9,000 bbl/day (800-1,400 cubic meters/day) in one embodiment, of 9,000-47,000 bbl/day (1,400-7,500 cubic meters/day) in another embodiment, and of 11,000-54,000 bbl/day (1,800-8,600 cubic meters/day) in still another embodiment. In some instances, the zero-flaring well testing assembly 12 can pump 0-120 million cubic feet (0-3.4 million cubic meters) of gas per day at standard conditions and 0-20,000 bbl (0-3,200 cubic meters) of oil per day at standard conditions.

In at least some embodiments, the one or more multiphase pumps 30 provide sufficient boost pressure to inject gas and oil from the well effluent as a mixture, which may also include water, into the output line 36 even when the wellhead pressure (e.g., at the tree 22) is less than the pressure within the output line 36 (e.g., production pressure in the case of a production line 36). Thus, the apparatus 20 and wellsite may also omit a gas compressor for pressurizing gas separated from the well effluent.

Performance of the multiphase pump 30 can depend on dynamic operational parameters, such as the GVF and gas and liquid flow rates of the pumped fluid. Exceeding the pump operating envelope of a multiphase pump can result in its malfunction or in process shut down. Keeping a multiphase pump 30 within its operating envelope can maximize performance while reducing risk of failure, risks to service quality, or other risks. In the case of a helicoaxial pump 30, for instance, the pressure developed by the helicoaxial pump 30 depends on the GVF of the pumped fluid. In some instances, such as cases in which the pumped fluid has a high GVF (e.g., a GVF above 90%, 95%, 96%, 97%, 98%, or 99%) during stable flow or in slugging conditions, the pressure developed by the helicoaxial pump 30 may be insufficient to deliver the pumped fluid into the output line 36. Condensate wells are an example of wells that may produce well effluent with such a high GVF.

High GVF values (e.g., higher than 90%) at the inlets of multiphase pumps 30 can create several problems, such as decrease of pressure developed by the pumps, increase of chances of pump failure, limitations on their maximum capacity, and placing the pumps outside their operating envelope. As discussed in greater detail below, the GVF of the well effluent can be measured and, in some cases, can be reduced by injecting liquid into the well effluent upstream of the multiphase pumps 30. This addition of liquid can allow the multiphase pumps 30 to be kept within their operating envelope while also allowing operation of the multiphase pumps 30 throughout a full range of GVF conditions (i.e., 0-100% GVF) of well effluent flowing into the zero-flaring well testing assembly 12 from the well. In at least some instances, the multiphase pumps 30 may also or instead be operated throughout a full range of water-liquid ratio (WLR) conditions (i.e., 0-100% WLR) of well effluent flowing into the zero-flaring well testing assembly 12 from the well.

In some embodiments, the GVF is monitored with the flowmeter 26. More specifically, the flowmeter 26 can be provided as a multiphase flowmeter 26, such as the Vx Spectra™ multiphase flowmeter produced by Schlumberger Ltd. The multiphase flowmeter 26 can be used to measure the flow rates of oil, water, and gas flowing through a pipe in real time without fluid separation or sampling. In service, the flowmeter 26 may be connected in line with the piping. The fluid mixture flows through the flowmeter 26, where the individual oil, water, and gas flow rates are measured. Outputs of the multiphase flowmeter 26 may include the total mass flow rate, the WLR at line conditions (i.e., the content of water inside the liquid phase), and the GVF at line conditions. Additional outputs may include oil, water, and gas flow rates at line conditions and other relevant ratios, such as gas-oil ratio (GOR). The multiphase flowmeter 26 may also provide real-time data transmission of the measured parameters and be used by a control system to detect minimum flow conditions and protect the system against surge.

Returning to FIG. 2, the multiphase flowmeter 26 can be used to measure the oil, gas, and water phase flow rates and volume fractions (e.g., GVF) at given pressure and temperatures for the well effluent. By positioning the multiphase flowmeter 26 upstream of the pumps 30, the GVF of the well effluent flowing toward the pumps 30 may be monitored. If the GVF exceeds a threshold level (e.g., 90%, 95%, 96%, 97%, 98%, or 99%), liquid may be added to the well effluent upstream of the pumps 30 to lower the GVF of the well effluent flowing into the pumps 30. In FIG. 2, the liquid added to the well effluent is delivered through a recirculation line 44 from the separator 32. More specifically, well effluent is pumped from the pumps 30 into the separator 32, and water separated from the well effluent in the separator 32 can be routed from a water outlet of the separator 32, via the recirculation line 44, and added to the well effluent at a location 46 upstream of the pumps 30. In other embodiments, liquid may be added to the well effluent in some other manner, such as by routing water (or some other liquid) from a sand filter or by injecting liquid (e.g., water or diesel) from an external source into the well effluent.

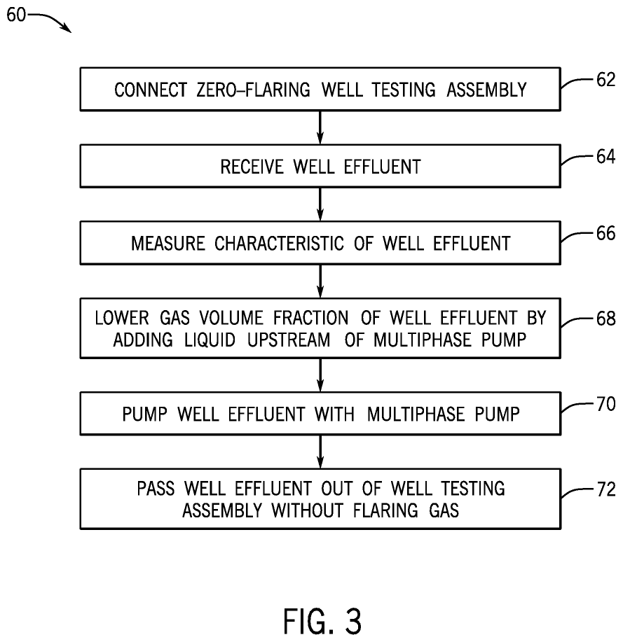

By way of further example, a process for using the zero-flaring well testing assembly 12 (e.g., the assembly depicted in FIG. 2) at a wellsite is generally represented by flowchart 60 in FIG. 3. In this embodiment, the zero-flaring well testing assembly 12 can be connected (block 62), such as in fluid communication between a wellhead assembly (e.g., tree 22) and an output line (e.g., output line 36), to receive a well effluent (block 64) that contains gas. One or more characteristics of the received gas-containing well effluent can be measured (block 66). Various characteristics can be measured, such as those described above, but in at least some instances the GVF of the well effluent is measured (e.g., in real time via a multiphase flowmeter 26). The multiphase flowmeter 26 installed upstream of the multiphase pumps 30 can provide data that enables quick response on changing dynamic conditions.

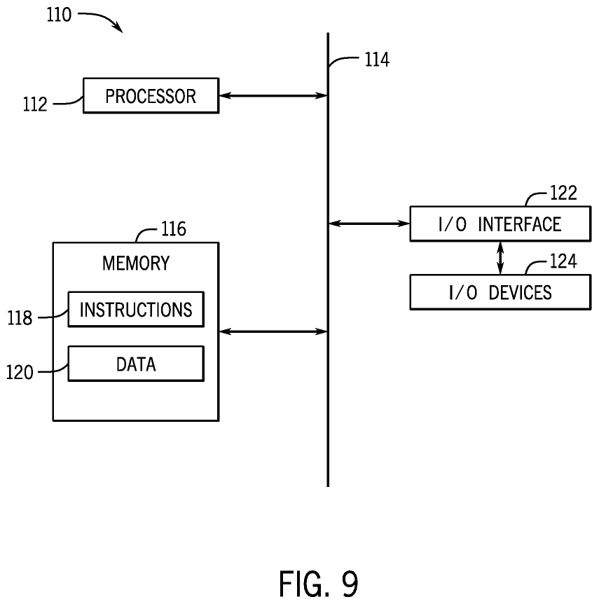

The process can also include lowering the GVF of the well effluent by adding liquid (block 68) upstream of the one or more multiphase pumps 30, such as by recirculating water from the separator 32 back into the well effluent at the location 46 upstream of the pumps 30. In some instances, a controller 110 (FIG. 9) of the zero-flaring well testing assembly 12 controls flow of the liquid to be added to a gas-containing well effluent based on the GVF or some other characteristic measured by the multiphase flowmeter 26. More specifically, a controller 110 can be programmed to cause flow of the liquid to be added to the gas-containing well effluent so as to lower the GVF of the gas-containing well effluent to no more than a threshold level in response to detection (e.g., via the multiphase flowmeter 26) that the GVF has exceeded the threshold. Any suitable threshold may be used, and this threshold may vary between embodiments, such as based on operating envelopes of the pumps 30 and other design considerations. In some embodiments, the threshold GVF level is set at or above 90%, 95%, 96%, 97%, 98%, or 99%. The threshold level may be predetermined before operation of the zero-flaring well testing assembly 12 or may be set or changed during operation.

In some embodiments, an inline choke (or other valve) can be installed at the water outlet of the separator 32, or between the water outlet and the location 46, to control flow of water from the separator 32 through the recirculation line 44. The controller 110 can automatically control opening and closing of the inline choke in response to GVF or other determined characteristics to supply sufficient additional liquid to the well effluent to maintain desired functioning of the multiphase pumps 30 (e.g., within their operating envelopes). Slugging of condensate wells is indicated by the detected increase of GVF and gas rates and decrease of liquid rates. In this situation, the inline choke may be opened to supply sufficient liquid volume for proper functioning of the pumps 30. In another instance, setting a higher frequency of the multiphase pumps 30 for extracting higher flowrates may make the pumps 30 more vulnerable to water packs that affect pump motor loading. Indication of increasing water flow rate (e.g., via the multiphase flowmeter 26) may be used to command (e.g., automatically via controller 110) reducing the water outlet inline choke diameter to maintain motor loading within an acceptable range.

Additionally, in the process of FIG. 3, the gas-containing well effluent may be pumped (block 70) through the zero-flaring well test assembly 12 with the one or more multiphase pumps 30. In at least some embodiments, the well effluent is then routed to a separator 32 downstream of the pumps 30. As noted above, water separated from the well effluent in the separator 32 may be recirculated to lower the GVF of the well effluent upstream of the pumps 30. Hydrocarbons (e.g., oil and gas) separated in the separator 32 can be routed from the separator 32 to the output line 36. In this manner, in the case of a gas-containing well effluent, at least a gas-containing portion of the well effluent is provided to the output line 36 without flaring the gas (block 72) or burning oil of the well effluent. In at least some embodiments, the separated oil and gas are combined downstream of the separator 32 before flowing into the output line 36.

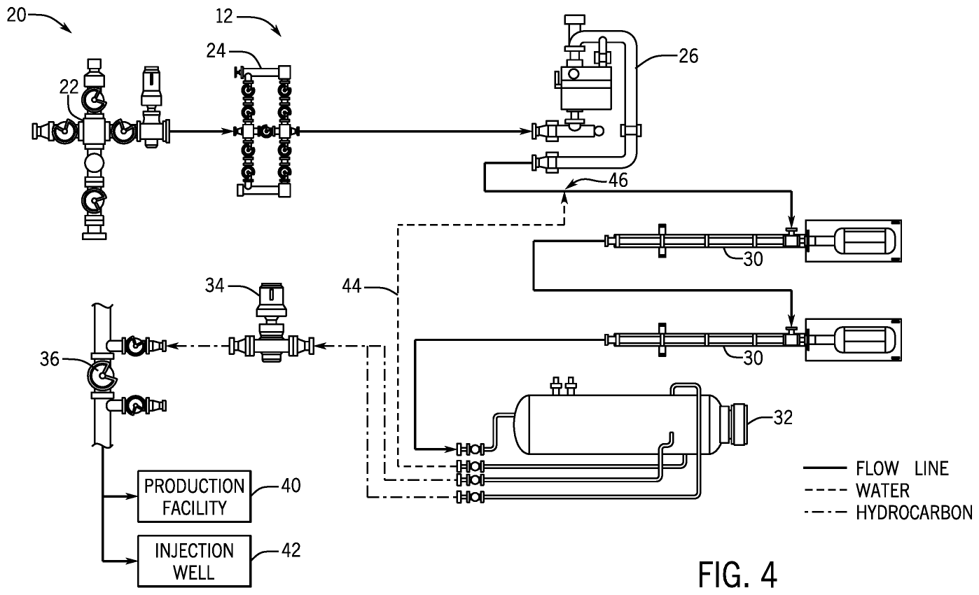

It will be appreciated that the techniques described above may be used with a zero-flaring well testing assembly 12 differing in configuration from that depicted in FIG. 2. For instance, different configurations of the zero-flaring well testing assembly 12 are depicted in FIGS. 4-7 in accordance with some embodiments. The zero-flaring well testing assembly 12 depicted in FIG. 4 is like that of FIG. 2 but with the pumps 30 connected in series, rather than in parallel. Connecting multiphase pumps 30 in series may provide increased head to the well effluent to facilitate injection into the output line 36 and increased production. In FIG. 4, the location 46 at which liquid from the recirculation line 44 may be added to the well effluent to reduce its GVF is downstream of the flowmeter 26. In at least some instances, the rate at which liquid (e.g., water from the separator 32) is added to the well effluent from the recirculation line 44 to lower the GVF before reaching the pumps 30 may be varied and controlled based on the GVF and flow rates measured with the multiphase flowmeter 26. If the measured GVF is below the threshold level, flow through the recirculation line 44 may be blocked (e.g., with the inline choke noted above). But when the measured GVF exceeds the threshold level, flow through the recirculation line 44 may be allowed to inject liquid into the well effluent at location 46 and reduce the GVF of the fluid pumped by the pumps 30. While certain examples of threshold values are provided above, in at least some cases the threshold value is also or instead the maximum recommended GVF operating value of the pumps 30.

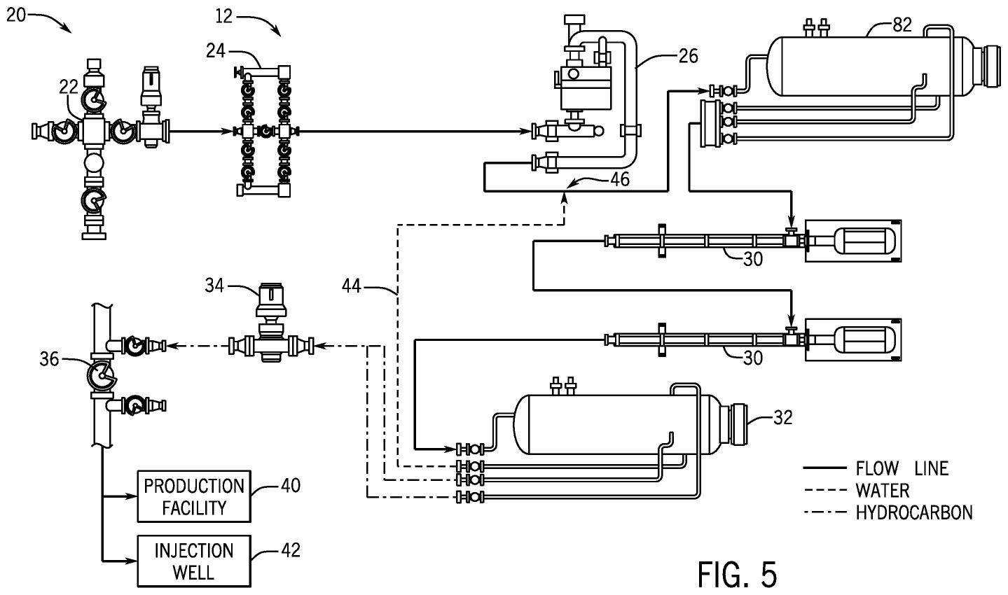

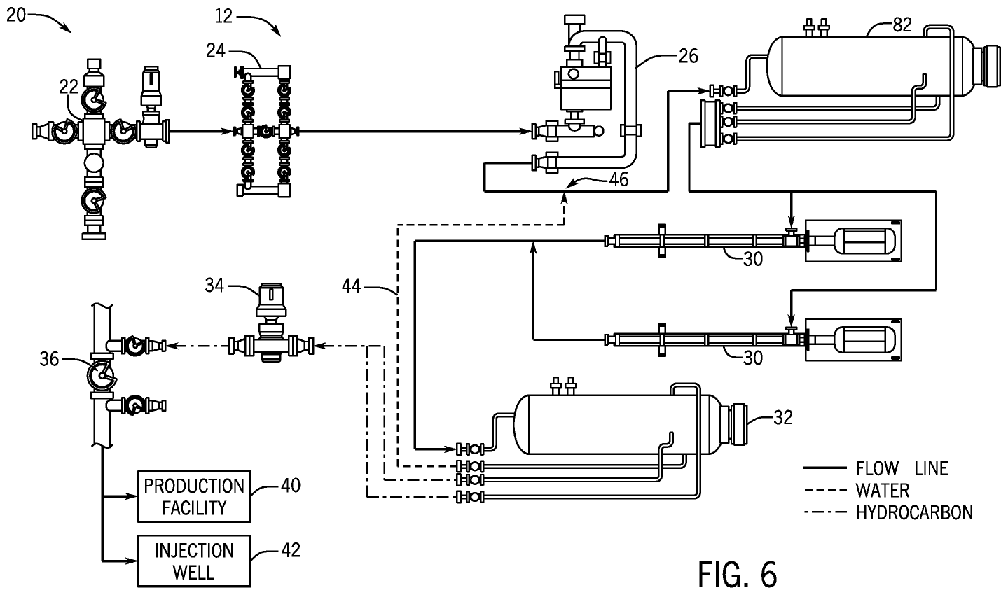

In FIGS. 5 and 6, the zero-flaring well testing assembly 12 includes a slug catcher 82 in addition to the other components described above for FIGS. 2 and 4. The slug catcher 82 may, in at least some cases, handle slugging behavior of the well, make the multiphase well effluent flow more homogenous, and achieve smoother performance of the one or more multiphase pumps 30. In FIG. 5, the pumps 30 are connected in series, while in FIG. 6 the pumps 30 are connected in parallel.

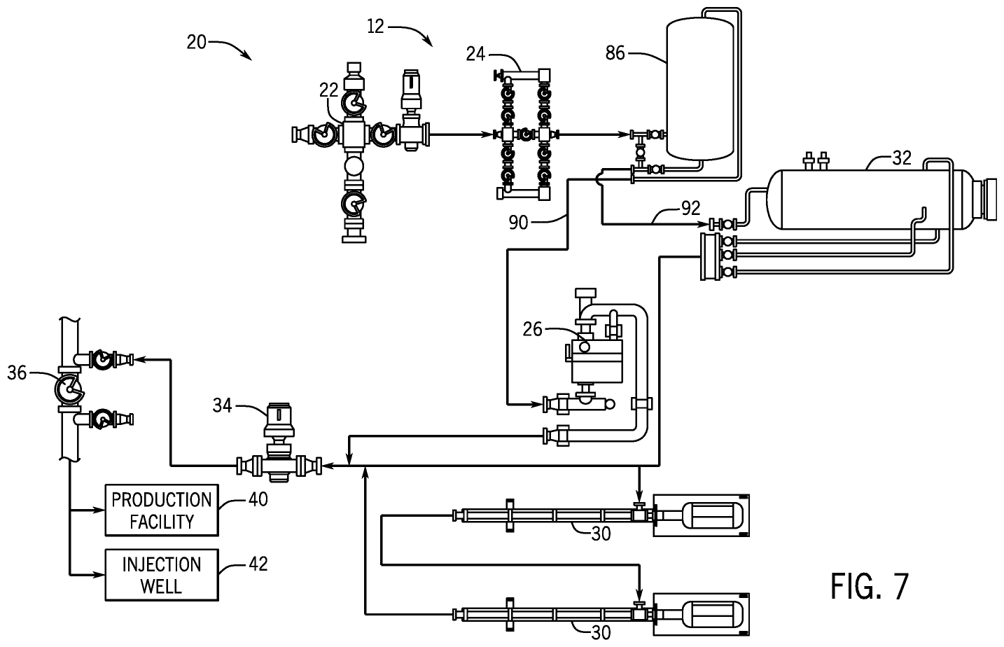

Another example of the zero-flaring well testing assembly 12 is depicted in FIG. 7 as having a scrubber 86 and a separator 32 upstream of the pumps 30. Well effluent from the manifold 24 may be routed into the scrubber 86. A first line 90 routes a portion of the well effluent from the scrubber 86 to the flowmeter 26 (which, again, may be a multiphase flowmeter 26), while a second line 92 routes another portion of the well effluent to the separator 32. In at least some instances, the fluid routed through the first line 90 is predominantly gas (but may also contain liquid), while the fluid routed through the second line 92 is predominantly liquid (but may also contain gas). The multiphase pumps 30 may be used to boost pressure and facilitate injection of well effluent into the outlet line 36.

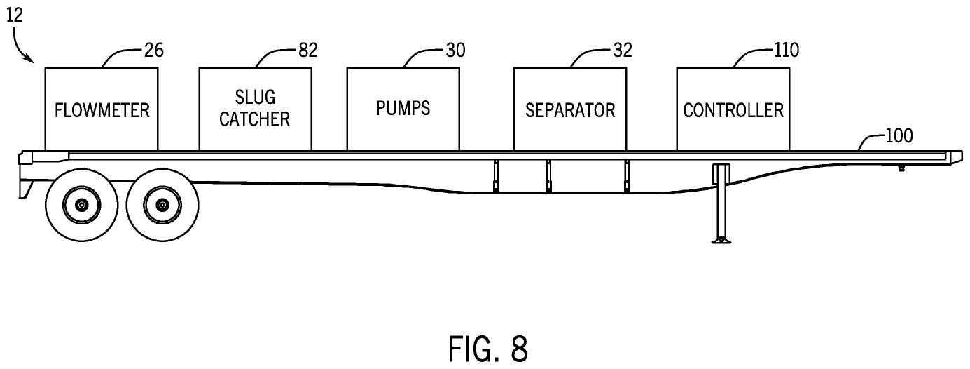

Some or all of the components of the zero-flaring well testing assembly 12 may be integrated in a mobile package to facilitate delivery to a wellsite. For example, the flowmeter 26, the pumps 30, and the separator 32 may be mounted together on a shared platform. Other components of the zero-flaring well testing assembly 12, such as various manifolds, the slug catcher 82, and the controller 110, can also or instead be mounted on the shared platform. In one embodiment generally depicted in FIG. 8, the shared platform is provided as a trailer 100, allowing the zero-flaring well testing assembly 12 to be towed to a wellsite.

Finally, a controller 110 for implementing various functionality described above can be provided in any suitable form. In at least some embodiments, such a controller 110 is provided in the form of a processor-based system, such as a personal computer, a handheld computing device, or a programmed logic controller. The controller 110 may be the flow computer of the multiphase flowmeter 26 in some instances. An example of such a processor-based controller 110 is generally depicted in FIG. 9. In this depicted embodiment, the controller 110 includes a processor 112 connected by a bus 114 to a memory device 116. It will be appreciated that the controller 110 could also include multiple processors or memory devices, and that such memory devices can include volatile memory (e.g., random-access memory) or non-volatile memory (e.g., flash memory or a read-only memory). The one or more memory devices 116 are encoded with application instructions 118 (e.g., software executable by the processor 112 to perform various functionality described above), as well as with data 120 (e.g., measured characteristics, pump specifications, pump operational data, GVF thresholds, and other data that facilitates control of the zero-flaring well testing assembly 12). For example, the application instructions 118 can be executed to monitor the GVF of well effluent upstream of the pumps 30 and selectively control injection of liquid to lower the GVF in accordance with techniques described above. In some instances, the application instructions 118 may be executed to automatically perform a procedure in response to measured characteristics, such as in response to detection of GVF above a desired threshold. In one embodiment, the application instructions 118 are stored in a read-only memory and the data 120 is stored in a writeable non-volatile memory (e.g., a flash memory).

The controller 110 also includes an interface 122 that enables communication between the processor 112 and various input or output devices 124. The interface 122 can include any suitable device that enables such communication, such as a modem or a serial port. The input and output devices 124 can include any number of suitable devices. For example, the devices 124 can include one or more sensors or meters (e.g., the flowmeter 26) for providing input to be used by the controller 110 to monitor and control operation of the zero-flaring well testing assembly 12. The devices 124 may also include a keyboard or other interface that allows user input to the controller 110, and a display, printer, or speaker to output information from the controller 110 to a user.

While the aspects of the present disclosure may be susceptible to various modifications and alternative forms, specific embodiments have been shown by way of example in the drawings and have been described in detail herein. But it should be understood that the invention is not intended to be limited to the particular forms disclosed. Rather, the invention is to cover all modifications, equivalents, and alternatives falling within the spirit and scope of the invention as defined by the following appended claims.

1. An apparatus comprising:
a wellhead assembly mounted on a well at a wellsite;
a production line to receive and transport a gas-containing well effluent; and
a zero-flaring well testing assembly coupled to receive the gas-containing well effluent from the wellhead assembly, wherein the zero-flaring well testing assembly includes a multiphase pump, and the zero-flaring well testing assembly is coupled to the production line so as to provide at least a gas-containing portion of the gas-containing well effluent received from the wellhead assembly by the zero-flaring well testing assembly to the production line.

2. The apparatus of claim 1, wherein the zero-flaring well testing assembly is configured to add liquid, at a location upstream of the multiphase pump, to the gas-containing well effluent received from the wellhead assembly.

3. The apparatus of claim 2, wherein the liquid that the zero-flaring well testing assembly is configured to add, at the location upstream of the multiphase pump, to the gas-containing well effluent received from the wellhead assembly includes water.

4. The apparatus of claim 3, wherein the zero-flaring well testing assembly includes a separator.

5. The apparatus of claim 4, wherein the liquid that the zero-flaring well testing assembly is configured to add, at the location upstream of the multiphase pump, to the gas-containing well effluent received from the wellhead assembly includes water separated from the gas-containing well effluent via the separator at a location downstream of the multiphase pump.

6. The apparatus of claim 3, wherein the zero-flaring well testing assembly includes a multiphase flowmeter.

7. The apparatus of claim 6, comprising a controller configured to control flow of the liquid added, at the location upstream of the multiphase pump, to the gas-containing well effluent received from the wellhead assembly based on a characteristic of the gas-containing well effluent measured by the multiphase flowmeter.

8. The apparatus of claim 7, wherein the controller is configured to, in response to detection via the multiphase flowmeter that a gas volume fraction of the gas-containing well effluent is above a threshold level, cause flow of the liquid added, at the location upstream of the multiphase pump, to the gas-containing well effluent to lower the gas volume fraction of the gas-containing well effluent to no more than the threshold level.

9. The apparatus of claim 1, wherein the zero-flaring well testing assembly includes an additional multiphase pump.

10. The apparatus of claim 9, wherein the multiphase pump and the additional multiphase pump are connected in series.

11. The apparatus of claim 1, wherein the apparatus does not include a gas flaring device at the wellsite to flare gas from the gas-containing well effluent.

12. An apparatus comprising:
a zero-flaring well testing assembly having a flowmeter, a multiphase pump, and a separator, wherein the zero-flaring well testing assembly is configured: to receive a gas-containing well effluent; to measure a characteristic of the gas-containing well effluent via the flowmeter; to separate water and gas from the gas-containing well effluent via the separator; to lower a gas volume fraction of the gas-containing well effluent upstream of the multiphase pump by adding the separated water to the gas-containing well effluent at a location of the zero-flaring well testing assembly upstream of the multiphase pump; and to route from the zero-flaring well testing assembly a gas-containing portion of the gas-containing well effluent received from the wellhead assembly into an output line without flaring the separated gas.

13. The apparatus of claim 12, wherein the flowmeter is a multiphase flowmeter upstream of the multiphase pump.

14. The apparatus of claim 12, wherein the characteristic of the gas-containing well effluent to be measured is the gas volume fraction of the gas-containing well effluent received in the zero-flaring well testing assembly.

15. The apparatus of claim 12, wherein the flowmeter, the multiphase pump, and the separator are mounted together on a shared platform.

16. The apparatus of claim 12, wherein the zero-flaring well testing assembly is a mobile well testing assembly installed on a trailer.

17. The apparatus of claim 12, wherein the zero-flaring well testing assembly includes a slug catcher.

18. A method comprising:
coupling a zero-flaring well testing assembly in fluid communication between a wellhead assembly and an output line at a wellsite, the zero-flaring well testing assembly including a flowmeter, a multiphase pump, and a separator;
receiving a gas-containing well effluent into the zero-flaring well testing assembly;
measuring a characteristic of the gas-containing well effluent via the flowmeter;
adding liquid to the gas-containing well effluent at a location upstream of the multiphase pump to lower a gas volume fraction of the gas-containing well effluent upstream of the multiphase pump; and
routing from the zero-flaring well testing assembly a gas-containing portion of the gas-containing well effluent into the output line without having flared any gas of the gas-containing well effluent received into the zero-flaring well testing assembly.

19. The method of claim 18, wherein adding the liquid to the gas-containing well effluent at the location upstream of the multiphase pump includes separating the liquid from the gas-containing well effluent with the separator at a location downstream of the multiphase pump and routing the separated liquid to the gas-containing well effluent at the location upstream of the multiphase pump.

20. The method of claim 18, comprising routing the gas-containing portion of the gas-containing well effluent to a production facility via the output line.

21. The method of claim 18, comprising injecting the gas-containing portion of the gas-containing well effluent into a well via the output line.
